(19) United States
(12) Patent Application Publication
(10) Pub. No.: US 2008/0108676 A1
(43) Pub. Date: May 8, 2008

(54) Benzothiazolecarboxamides

(75) Inventors: William Brown, Montreal (CA); Andrew Griffin, Montreal (CA); Shawn Johnstone, Montreal (CA); Denis Labrecque, Montreal (CA); Alexander Munro, Montreal (CA); Sandrine Pache, Montreal (CA); Christopher Walpole, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: New Benzothiazolecarboxamides, Sodertalje (SE)

(21) Appl. No.: 11/721,635

(22) PCT Filed: Dec. 19, 2005
(86) PCT No.: PCT/SE05/01964

§ 371 Date: Jun. 13, 2007

(30) Foreign Application Priority Data
Dec. 21, 2004 (SE) 0403117-5

(51) Int. Cl. A61K 31/428; C07D 277/62; A61P 11/00; A61P 25/00

(52) U.S. Cl. 514/367; 548/180

(57) ABSTRACT

The present invention relates to new compounds of formula I, (I) wherein R¹ to R⁴, m, n and p, are as defined as in formula I, or salts, solvates or solvated salts thereof, processes for their preparation and to a new intermediate used in the preparation thereof, pharmaceutical formulations containing said compounds and to the use of said compounds in therapy.

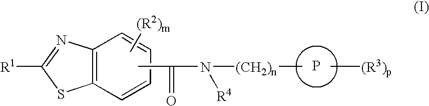

### FIELD OF THE INVENTION

The present invention relates to new compounds, to pharmaceutical compositions containing said compounds and to the use of said compounds in therapy. The present invention further relates to processes for the preparation of said compounds and to new intermediate used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., et al., et. al. Nature (1997) v.389 p 816-824). Functional studies using VR1 indicate that it is also activated by noxious heat, tissue acidification) and other inflammatory mediators (Tominaga, M., et. al. Neuron (1998) v.21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects.

Compounds with VR1 inhibitor activity are believed to be of potential use for the treatment and/or prophylaxis of disorders such as pain, especially that of inflammatory or traumatic origin such as arthritis, ischaemia, fibromyalgia, low back pain and post-operative pain (Walker et al., J Pharmacol Exp Ther. (2003) January; 304(1):56-62). In addition to this visceral pains such as chronic pelvic pain, cystitis, irritable bowel syndrome (IBS), pancreatitis and the like, as well as neuropathic pain such as sciatica, diabetic neuropathy, HIV neuropathy, multiple sclerosis, and the like (Walker et al ibid, J Pharmacol Exp Ther. (2003) March; 304(3):940-8), are potential pain states that could be treated with VR1 inhibition. These compounds are also believed to be potentially useful for inflammatory disorders like asthma, cough, inflammatory bowel disease (IBD) (Hwang, et al., Curr Opin Pharmacol (2002) June; 2(3):235-42). Compounds with VR1 blocker activity are also useful for itch and skin diseases like psoriasis and for gastro-esophageal reflux disease (GERD), emesis, urinary incontinence and hyperactive bladder (Yiangou et al BJU Int (2001) June; 87(9):774-9, Szallasi, Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

The role for VR1 antagonists in Inflammatory Bowel Diseases (IBD) is further supported by the finding that primary sensory neuron denervation by subcutaneous administration of capsaicin to neonatal rats, resulted in decreased levels of disease activity index (DAI), MPO and histological damage to the gut in DSS colitis model compared to control (N Kihara, et al., Gut, 2003. 52: p. 713-719). TRPV1 antagonists attenuate macroscopic symptoms in DSS colitis model in mice (E. S. KIMBALL, et al., Neurogastroenterol Motil, 2004. 16: p. 1-8).

The potential for a role for VR1 antagonists in Irritable Bowel Syndrome (IBS) has been described. Patients with fecal urgency and rectal hypersensitivity have increased levels of TRPV1 expression in nerve fibres in muscle, submucosal and mucosal layers. This also correlates with increase sensitivity to heat and distension (C L H Chan, et al., THE LANCET, 2003. 361 (February 1): p. 385-91). Jejunal wide dynamic range (WDR) afferents show lower firing in response to pressure ex vivo in TRPV1−/− mice (Rong W, H. K., et al., J Physiol (Lond). 2004. 560: p. 867-881). The visceromotor responses to jejunal and colorectal distension in rat are affected by a TRPV1 antagonist using both ramp and phasic distensions (Winchester, E M G response to jejunal and colorectal distension in rat are affected by a TRPV1 antagonist in both ramp and phasic distensions. DDW abstract, 2004). Capsaicin applied to the ileum induce pain and mechanical hyperalgesia in human experimental model (Asbjøm Mohr Drewes, et al., Pain, 2003. 104: p. 333-341).

A role in Gastroesophageal Reflux Disease (GERD) for VR1 antagonists has been mentioned in the literature. Patients with oesophagitis have increased levels of TRPV1 expression in peripheral nerves enervating the oesophageal epithelium (P. J. Matthews, et al., European J. of Gastroenterology & Hepatology, 2004. 16: p. 897-902). Even if the TRPV1 antagonist JYL1421 only has minor effects of acid-induced excitation of esophageal afferents, an antagonist with a different profile has yet to be evaluated. Since TRPV1 appears to play a role in mechanosensation, it is possible that antagonists may inhibit TLESRs, the main cause of gastroesophageal reflux.

A further potential use relates to the treatment of tolerance to VR1 activators.

VR1 inhibitors may also be useful in the treatment of interstitial cystitis and pain related to interstitial cystitis.

### DETAILED DESCRIPTION OF THE INVENTION

The object of the present invention is to provide compounds exhibiting an inhibitory activity at the vanilloid receptor 1 (VR1).

The present invention provides a compound of formula I

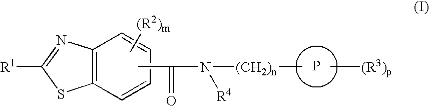

wherein:
ring P is C₆₋₁₀-aryl, C₃₋₁₁cycloalkyl or C₅₋₁₀heteroaryl;

### R¹ is H, C₁₋₄alkyl, hydroxyC₁₋₆alkyl, C₁₋₆alkylOC₀₋₆alkyl, COOC₀₋₆alkyl, NH₂, NHC₁₋₆alkyl, N(C₁₋₆alkyl)₂, NH(aryl) or N(aryl)₂;

### R² is H, C₁₋₄alkyl, halo, hydroxyC₀₋₆alkyl or C₁₋₆alkylOC₀₋₆alkyl;

m is 0, 1, 2 or 3;
n is 0, 1, 2, 3, 4 or 5;
R³ is NO₂, NH₂C₀₋₆alkyl, halo, N(C₁₋₆alkyl)₂C₀₋₆alkyl, C₁₋₆alkyl, C₂₋₆alkenyl, C₂₋₆alkynyl, C₁₋₆haloalkyl, C₁₋₆haloallylO, C₅₋₆arylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl,

### C₁₋₆alkylNC₀₋₆alkyl, (C₀₋₆alkyl)₂NC(O)C₀₋₆alkyl, (C₀₋₆alkyl)₂OC(O)C₀₋₆alkyl or (C₀₋₆alkyl)₂C(O)OC₀₋₆alkyl;

p is 1, 2, 3, 4 or 5; and

### R⁴ is H, C₁₋₆alkyl, arylC₀₋₆alkyl, C₁₋₆alkylOC₀₋₆alkyl or N(C₁₋₆alkyl)₂C₀₋₆alkyl,

or salts, solvates or solvated salts thereof.

One embodiment of the invention relates to the compound of formula Ib wherein R¹, R³, m and p, are as defined above, and n is 0 and R² and R⁴ are H.

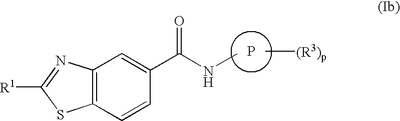

One embodiment of the invention relates to the compound of formula Ic, wherein R¹, R³, m and p, are as defined above, and n is 1, 2, 3, 4 or 5 and R² and R⁴ are H.

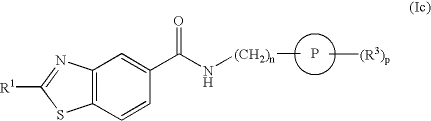

In a further embodiment of the invention P is phenyl.

In yet another embodiment of the invention R¹ is methyl or hydroxyC₁₋₃alkyl. In one embodiment R¹ is methyl, hydroxymethyl, hydroxyethyl or hydroxypropyl.

In another embodiment n is 0, 1 or 2.

In yet a further embodiment R³ is halo, C₁₋₃alkyl, C₁₋₃haloalkyl, C₅₋₆aryl, C₁₋₂alkylO or (C₀₋₆alkyl)₂NC(O)C₀₋₆alkyl.

In another embodiment R³ is tert-butyl, phenyl, fluoromethyl, difluoromethyl or trifluoromethyl.

One embodiment of the invention relates to compounds selected from the group consisting of
N-4-tert-butylphenyl-2-methyl-1,3-benzothiazole-5-carboxamide,
N-4-cyclohexylphenyl-2-methyl-1,3-benzothiazole-5-carboxamide,
2-methyl-N-[2-methyl-4-trifluoromethylphenyl]-1,3-benzothiazole-5-carboxamide,
2-methyl-N-[4-trifluoromethylphenyl]-1,3-benzothiazole-5-carboxamide,
2-methyl-N-[3-trifluoromethylphenyl]-1,3-benzothiazole-5-carboxamide,
2-methyl-N-[2-trifluoromethylbenzyl]-1,3-benzothiazole-5-carboxamide,
2-methyl-N-[4-trifluoromethylbenzyl]-1,3-benzothiazole-5-carboxamide,
2-methyl-N-[3-trifluoromethylbenzyl]-1,3-benzothiazole-5-carboxamide,
N-4-methoxy-2-naphthyl-2-methyl-1,3-benzothiazole-5-carboxamide,
N-4-tert-butylphenyl-2-hydroxymethyl-1,3-benzothiazole-5-carboxamide,
N-(4-bromophenyl)-2-methyl-1,3-benzothiazole-5-carboxamide,
2-methyl-N-[2-(4-methylphenyl)ethyl]-1,3-benzothiazole-5-carboxamide,
N-[2-(3-fluorophenyl)ethyl]-2-methyl-1,3-benzothiazole-5-carboxamide,
N-(5-isopropoxy-1-naphthyl)-2-methyl-1,3-benzothiazole-5-carboxamide,
2-methyl-N-{2-[4-(trifluoromethyl)phenyl]ethyl}-1,3-benzothiazole-5-carboxamide,
N-[2-(4-ethylphenyl)ethyl]-2-methyl-1,3-benzothiazole-5-carboxamide,
N-[2-(4-fluorophenyl)ethyl]-2-methyl-1,3-benzothiazole-5-carboxamide,
N-[2-(4-tert-butylphenyl)ethyl]-2-methyl-1,3-benzothiazole-5-carboxamide,
N-[2-(4-methoxyphenyl)ethyl]-2-methyl-1,3-benzothiazole-5-carboxamide,
N-(4-isopropylphenyl)-2-methyl-1,3-benzothiazole-5-carboxamide,
N-[2-(4-chlorophenyl)ethyl]-2-methyl-1,3-benzothiazole-5-carboxamide,
N-[2-(3,4-dichlorophenyl)ethyl]-2-methyl-1,3-benzothiazole-5-carboxamide,
N-4-tert-butylphenyl-2-hydroxymethyl-1,3-benzothiazole-5-carboxamide,
2-(hydroxymethyl)-N-[2-(4-methylphenyl)ethyl]-1,3-benzothiazole-5-carboxamide, and
N-[2-(3-fluorophenyl)ethyl]-2-(hydroxymethyl)-1,3-benzothiazole-5-carboxamide
   or salts, solvates or solvated salts thereof.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term C₁₋₃ alkyl having 1 to 3 carbon atoms and may be methyl, ethyl, n-propyl or i-propyl.

The term ‘C₀’ means “a bond” or “does not exist”. For example when R³ is C₀alkyl, R³ is a bond and “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alynyl” having 2 to 6 carbon atoms and one or two triple bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

The term “heterocycloalkyl” denotes a 3- to 7-membered, non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon unsaturated aromatic ring system. Examples of “aryl” may be, but are not limited to phenyl and naphthyl.

In this specification, unless stated otherwise, the term “heteroaryl” refers to an optionally substituted monocyclic or bicyclic ring system whereby at least one ring is aromatic independently from N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl or oxazolyl.

In this specification, unless stated otherwise, the terms “heteroarylalkyl” and “phenylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

Unless specified otherwise within this specification, the nomenclature used in this specification generally follows the examples and rules stated in Nomenclature of Organic Chemistry, Sections A, B, C, D, E, F, and H. Pergamon Press, Oxford, 1979, which is incorporated by references herein for its exemplary chemical structure names and rules on naming chemical structures.

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Medical Use

Surprisingly, it has been found that the compounds according to the present invention are useful in therapy. The compounds of formula I, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed the peripheral nervous system and in other tissues. Thus, it is expected that the compounds of the invention are well suited for the treatment of VR1 mediated disorders.

The compounds of formula I are expected to be suitable for the treatment of acute and chronic pain, acute and chronic neuropathic pain and acute and chronic inflammatory pain. Examples of such disorder may be selected from the group comprising arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischaemic and angina, neuropathic pain disorders such as diabetic neuropathy, HIV neuropathy, chemotherapy induced neuropathies, post-herpetic neuralgia, post traumatic neuralgia and complex regional syndrome as well as itch.

Further relevant disorders may be selected from the group comprising gastro-esophageal reflux disease (GERD), functional gastrointestinal disorders (FGD) such as irritable bowel syndrome (IBS), irritable bowel syndrome (IBS), and functional dyspepsia (FD). Further examples of disorders are overactive bladder (“OAB”), a term for a syndrome that encompasses urge incontinence, urgency and frequency. Compounds of the invention may alleviate urinary incontinence (“UI”) the involuntary loss of urine that results from an inability of the bladder to retain urine as a consequence of either urge (urge incontinence), or physical or mental stress (stress incontinence).

Other relevant disorders may be psoriasis, and emesis.

Yet further relevant disorders are related to respiratory diseases and may be selected from the group comprising cough, asthma, chronic obstructive lung disease and emphysema, lung fibrosis and interstitial lung disease.

The VR1 inhibitor(s) for respiratory use, may be administrated by either an oral or inhaled route. The respiratory disease may be an acute and chronic illness and may be related to infection(s) and/or exposure to environmental pollution and/or irritants.

The compounds of formula I may also be used as antitoxin to treat (over-) exposure to VR1 activators like capsaicin, tear gas, acids or heat. Regarding heat, there is a potential use for VR1 antagonists in (sun-) burn induced pain, or inflammatory pain resulting from burn injuries.

The compounds may further be used for treatment of tolerance to VR1 activators.

One embodiment of the invention relates to the use of the compounds of formula I as hereinbefore defined, in therapy.

Another embodiment of the invention relates to the use of the compounds of formula I as hereinbefore defined, for treatment of VR1 mediated disorders.

A further embodiment of the invention relates to the use of the compounds of formula I as hereinbefore defined, for treatment of acute and chronic pain.

Yet another embodiment of the invention relates to the use of the compounds of formula I as hereinbefore defined, for treatment of acute and chronic neuropathic pain.

Yet a further embodiment of the invention relates to the use of the compounds of formula I as hereinbefore defined, for treatment of acute and chronic inflammatory pain.

One embodiment of the invention relates to the use of the compounds of formula I as hereinbefore defined, for treatment of arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischaemic and angina, neuropathic pain disorders such as diabetic neuropathy, HIV neuropathy, chemotherapy induced neuropathies, post-herpetic neuralgia, post traumatic neuralgia and complex regional syndrome as well as itch.

Another embodiment of the invention relates to the use of the compounds of formula I as hereinbefore defined, for treatment of gastro-esophageal reflux disease, functional gastrointestinal disorders, irritable bowel syndrome, irritable bowel syndrome and functional dyspepsia.

A further embodiment of the invention relates to the use of the compounds of formula I as hereinbefore defined, for treatment of overactive bladder.

Yet a further embodiment of the invention relates to the use of the compound of formula I as hereinbefore defined, for the treatment of respiratory diseases selected from the group comprising of cough, asthma, chronic obstructive lung disease and emphysema, lung fibrosis and interstitial lung disease.

One embodiment of the invention relates to the use of the compound of formula I as hereinbefore defined, in the manufacture of a medicament for treatment of VR1 mediated disorders and for treatment of acute and chronic pain, acute and chronic neuropathic pain and acute and chronic inflammatory pain, and respiratory diseases, and any other disorder mentioned above.

Another embodiment of the invention relates to a method of treatment of VR1 mediated disorders and acute and chronic pain, acute and chronic neuropathic pain and acute and chronic inflammatory pain, and respiratory diseases, and any other disorder mentioned above, comprising administering to a mammal, including man in need of such treatment, a therapeutically effective amount of the compounds of formula I, as hereinbefore defined.

A further embodiment of the invention relates to a pharmaceutical composition comprising a compound of formula I as hereinbefore defined, for use in treatment of VR1 mediated disorders and for treatment of acute and chronic pain, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

### Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Examples of Pharmaceutical Composition

The following illustrate representative pharmaceutical dosage forms containing a compound of formula I, or salts, solvates or solvated salts thereof, (hereafter compound X) for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | mg/capsule |
|---|---|
| Compound X | 10 |
| Lactose | 488.5 |
| Magnesium stearate | 1.5 |

| (c): Injection | (50 mg/ml) |
|---|---|
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above compositions may be obtained by conventional procedures well known in the pharmaceutical art.

### Methods of Preparation

### General Methods of Preparation

One embodiment of the invention relates to a process for the preparation of the compound of formula I, wherein R¹ to R⁴, m, n and p, are as defined above, comprising;

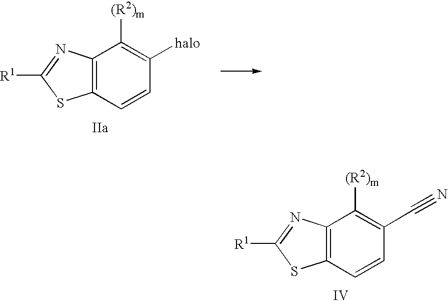

a-i) cyanidation of compound of formula IIa through metal halogen exchange.

This reaction may be performed in any manner known to the skilled person in the art. Cyanide formation may be performed via palladium catalyzed reaction with zinc cyanide.

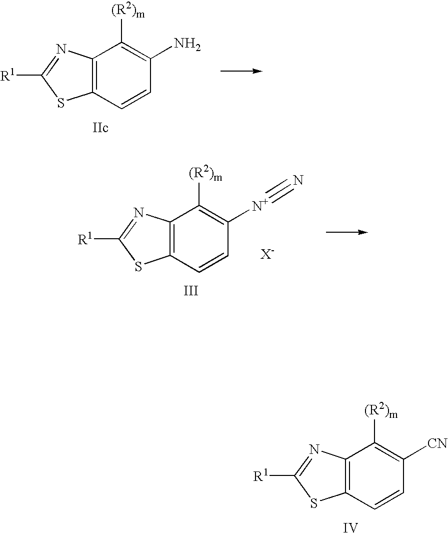

a-ii) Reaction of an aromatic amine of formula (IIc) with sodium nitrite in the presence of an acid like HCl, H₂SO₄ or TFA, to obtain a diazonium intermediate (III), that is reacted in-situ with sulphur dioxide or in the presence of copper chloride to give cyanide of formula IV.

This reaction may be performed in any manner known to the skilled person in the art. Suitable solvents to be used for this reaction may be water, acetone, organic acids such as acetic acid and TFA, or mixtures thereof. The temperature may be between 0 and 10° C. and the reaction time may be between 0.5 and 30 h.

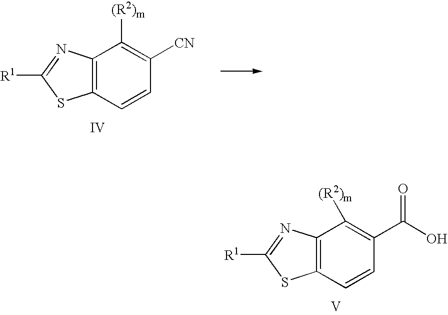

b) Hydrolysis of an aromatic cyanide of formula IV to obtain the carboxylic acid of formula V.

This reaction may be performed in any manner known to the skilled person in the art. Under acidic conditions, suitable solvents may be water, hydrochloridric acid, sulphuric acid, or any mixtures thereof. Alternatively, it can be done in basic conditions by reaction with a suitable inorganic base in water or organic solvents like methanol, ethanol, iso-propanol or tert-butanol, or mixtures thereof. The temperature may be between 70 and 100° C.

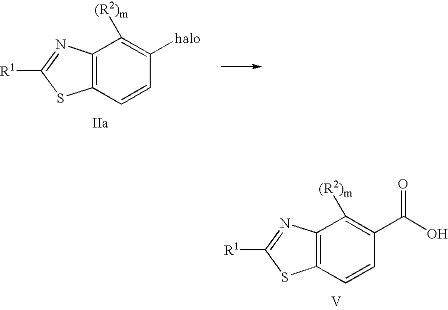

c) A metal halogen exchange followed by carbonylation with carbone dioxide to obtain the compound of formula V.

This reaction may be performed in any manner known to the skilled person in the art. Metal halogen exchange may be achieved with alkyl lithium or dialkyl magnesium Suitable solvents to be used for this reaction may be ethers such as ethyl ether, tetrahydrofuran and dioxin, or any mixtures thereof. The temperature may be between −60 and −70° C. and the reaction time may be between 1 and 3 h. The lithium or magnesium species may be reacted with carbon dioxide as gas or solid.

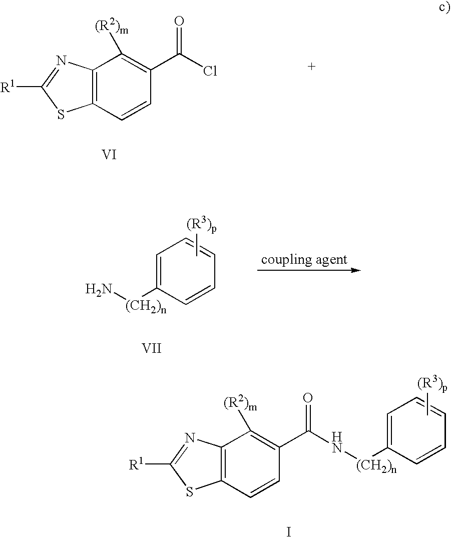

d) reaction of the aromatic acyl chloride of formula VI with properly substituted amines of formula VII.

This reaction may be performed in any manner known to the skilled person in the art. Suitable solvents to be used for this reaction may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or any mixtures thereof. Catalysts such as heteroaromatic bases like pyridine and lutidine or tertiary amines like triethylamine, N-methylmorpholine and ethyl diisopropylamine may be used as well. The temperature may be between 10 and 60° C. and the reaction time may be between 3 and 30 h.

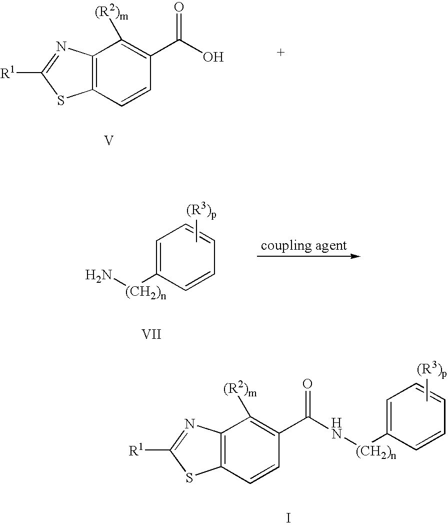

e) Reaction of the carboxylic acids of formula V with the aromatic amine of formula VII. Suitable solvents to be used for this reaction may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxin, or any mixtures thereof. Catalysts such as heteroaromatic bases like pyridine and lutidine or tertiary amines like triethylamine, N-methylmorpholine and ethyl diisopropylamine may be used as well. The temperature may be between 10 and 60° C. and the reaction time may be between 3 and 30 h.

### Intermediates

A further embodiment of the invention relates to compound 2-methyl-1,3-benzothiazole-5-carboxylic acid,

which may be used as intermediate in the preparation of compounds suited for the treatment of VR1 mediated disorders, especially for use as intermediate for the preparation of compounds of formula I.

### EXAMPLES

The invention will now be illustrated by the following Examples in which, generally
(i) operations were carried out at ambient or room temperature, i.e. in the range 17 to 25° C. and under an atmosphere of an inert gas such as argon unless otherwise stated;
(ii) evaporations were carried out by rotary evaporation in vacuo and work-up procedures were carried out after removal of residual solids by filtration;
(iii) column chromatography (by the flash procedure) was performed on Silicycle silica gel (grade 230-400 mesh, 60 Å, cat. Numb. R10030B) or obtained from Silicycle, Quebec, Canada or high pressure liquid chromatography (HPLC) was performed on C18 reverse phase silica, for example on a Phenomenex, Luna C-18 100 Å preparative reversed-phase column;
(iv) the ¹H NMR spectra were recorded on a Varian or Brucker at 400 or 600 MHz.
(v) the mass spectra were recorded utilising electrospray (LC-MS; LC:Waters 2790, column XTerra MS C₈2.5 μm 2.1×30 mm, buffer gradient H₂O+0.1% TFA:CH₃CN+0.04% TFA, MS: micromass ZMD//ammonium acetate buffer) ionisation techniques;
(vi) yields, where present, are not necessarily the maximum attainable;
(vii) intermediates were not necessarily fully purified but their structures and purity were assessed by thin layer chromatographic, HPLC and/or NMR analysis;
(viii) the following abbreviations have been used:
HPLC high performance liquid chromatography
LC liquid chromatography
MS mass spectrometry
ret. time retention time
AcCl acetyl chloride
DCM dichloromethane
DMAP dimethylaminopyridine
DMF dimethylformamide
EtOH ethanol
EtOAc ethyl acetate
EDC 1-(3-dimethylaminopropyl)-3-ethylcarbodiimide hydrochloride
HATU O-(7-azabenzotriazol-1-yl)-N,N,N′,N′-tetramethyluronium
Hexafluorophosphate
HCl hydrochloric acid
MeOH methanol
THF tetrahydrofurane

### Intermediate 1

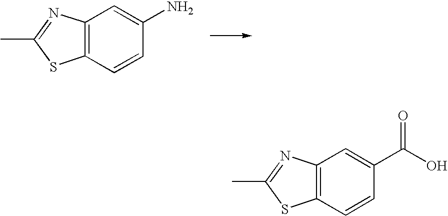

### 2-methyl-1,3-benzothiazole-5-carboxylic acid

A solution of 2-methyl-5-aminobenzothiazole (10.0 g, 61.1 mmol) in acetone (250 mL) was cooled to 0° C., and concentrated HCl (13.5 mL) was added. A solution of NaNO₂ (5.22 g, 75.7 mmol) in water (75.0 mL) was added in one portion to the first solution. The resulting mixture was stirred for 3 minutes, and a solution of KI (20.4 g, 123 mmol) in water (75.0 mL) was added. The mixture was stirred for an additional 10 minutes and then concentrated under reduced pressure to yield a residue, which was dissolved in a 9:1 mixture of DCM and MeOH and washed with a saturated solution of NaHCO₃. The organic fraction was washed with brine, dried with Na₂SO₄, filtered, concentrated under reduced pressure and dried under high vacuum. The resulting iodide, ZnCN₂ (7.17 g, 61.1 mmol) and Pd(PPh₃)₄ (2.00 g, 1.73 mmol) were mixed in DMF (200 mL) and heated to 100° C. for 12 hours, under a N₂ atmosphere. The solution was then cooled to room temperature, and the solvent was evaporated under reduced pressure. The residue was dissolved in DCM and washed with a saturated solution of NaHCO₃ followed by brine. The organic phase was dried with Na₂SO₄, filtered and concentrated under reduced pressure to yield the nitrile. ¹H NMR (400 MHz, CHLOROFORM-D) δ ppm 2.89 (s, 3H) 7.58 (dd, J=8.40, 1.56 Hz, 1H) 7.93 (d, J=8.20 Hz, 1H) 8.22 (d, J=0.98 Hz, 1H); MS [M+] calcd. 174.0, found 174.8. A solution of the nitrile in 6.70 N HCl (150 mL) was refluxed for 12 hours. The solution was cooled to room temperature and then concentrated under reduced pressure. The product was purified by flash chromatography on reverse phase silica gel eluting with mixtures of EtOH and water (15/85 to 90/10) (4.45 g, 19.5 mmol, 32% for 3 steps). ¹H NMR (600 MHz, DMSO-D6) δ ppm 2.81 (s, 3H) 7.92 (d, J=8.45 Hz, 1H) 8.14 (d, J=8.45 Hz, 1H) 8.38 (s, 1H); MS [M+] cacld. 193.0, found 193.8.

### Examples 1

### N-4-tert-butylphenyl-2-methyl-1,3-benzothiazole-5-carboxamide

2-Methyl-1,3-benzothiazole-5-carboxylic acid (90.0 mg, 0.400 mml) was dissolved in DMF (3.00 mL), and HATU (190 mg, 0.500 mmol), 4-tert-butylaniline (75.0 mg, 0.500 mmol) and Et₃N (0.100 mL) were added. The mixture was stirred for 3 hours, and the solvents were evaporated. The product was purified by flash chromatography on silica gel eluting with mixtures of hexane and EtOAc (9:1 to 4:1) to yield the product (42.0 mg, 0.129 mmol, 32.0%). ¹H NMR (400 MHz, DMSO-D6) δ ppm 1.27 (s, 9H) 2.83 (s, 3H) 4.90-5.18 (br s, 1H) 7.36 (d, J=8.98 Hz, 2H) 7.71 (dd, J=8.98, 2.73 Hz, 2H) 7.96 (dd, J=8.40, 1.76 Hz, 1H) 8.16 (d, J=8.40 Hz, 1H) 8.51 (d, J=1.37 Hz, 1H) 10.31 (s, 1H); MS [M+H] calcd. 325.0, found 325.0.

### Examples 2

### N-4-cyclohexylphenyl-,2-methyl-1,3-benzothiazole-5-carboxamide

2-Methyl-1,3-benzothiazole-5-carboxylic acid (100 mg, 0.440 mml) was dissolved in DMF (5.00 mL), and HATU (190 mg, 0.500 mmol), 4-cyclohexylaniline (88.0 mg, 0.500 mmol) and Et₃N (0.100 mL) were added. The mixture was stirred for 3 hours, and the solvents were evaporated. The product was purified by flash chromatography on silica gel eluting with mixtures of hexane and EtOAc (9:1 to 4:1) to yield a mostly pure product, which was recrystallized from heptanes and EtOAc to yield a pure product (15.1 mg, 0.043 mmol, 10.0%). ¹H NMR (400 MHz, DMSO-D6) δ ppm 1.15-1.50 (m, 5H) 1.60-1.83 (m, 6H) 2.82 (s, 3H) 7.18 (d, J=8.59 Hz, 2H) 7.67 (d, J=8.59, 2H) 7.94 (dd, J=8.40, 1.76 Hz, 1H) 8.14 (d, J=8.40 Hz, 1H) 8.48 (d, J=1.56 Hz, 1H) 10.30 (s, 1H); MS [M+] calcd. 350.2, found 351.0.

### Examples 3

### 2-methyl-N-[2-methyl-4-trifluoromethylphenyl]-1,3-benzothiazole-5-carboxamide

2-Methyl-1,3-benzothiazole-5-carboxylic acid (90.0 mg, 0.470 mmol) was mixed with 2-methyl-4-trifluoromethylaniline (123 mg, 0.700 mmol), EDC (134 mg, 0.700 mmol) and DMAP (85.0 mg, 0.700 mmol) in DCM (5.00 mL) and DMF (3.00 mL) for 48 hours. The mixture was concentrated, and the product was purified by flash chromatography on silica gel, eluting with mixtures of heptanes and EtOAc (95/5 to 75/25), to yield the product (14.0 mg, 0.0400 mmol, 8.50%). ¹H NMR (600 MHz, CHLOROFORM-D) δ ppm 2.42 (s, 3H) 2.89 (s, 3H) 7.50 (s, 1H) 7.54 (d, J=8.45 Hz, 1H) 7.85-8.05 (m, 3H) 8.32 (d, J=8.45 Hz, 1H) 8.41 (s, 1H); MS [M+H] calcd. 351.0, found 351.0.

### Examples 4

### 2-methyl-N-[4-trifluoromethylphenyl]-1,3-benzothiazole-5-carboxamide

2-Methyl-1,3-benzothiazole-5-carboxylic acid (150 mg, 0.660 mmol) was mixed with 4-trifluoromethylaniline (209 mg, 1.30 mmol), EDC (249 mg, 1.30 mmol) and DMAP (158 mg, 1.30 mmol) in DCM (5.00 mL) and DMF (2.00 mL) for 18 hours. The mixture was concentrated, and the product was purified by flash chromatography on silica gel, eluting with mixtures of heptanes and EtOAc (95/5 to 0/100), to yield the product (111 mg, 0.329 mmol, 50.0%). ¹H NMR (600 MHz, CHLOROFORM-D) δ ppm 2.85 (s, 3H) 7.55 (d, J=8.45 Hz, 2H) 7.84 (d, J=8.45 Hz, 2H) 7.94 (dd, J=8.45, 1.79 Hz, 1H) 8.04 (d, J=8.45 Hz, 1H) 8.38 (d, J=1.02 Hz, 1H); MS [M+H] calcd. 337.0, found 337.0.

### Examples 5

### 2-methyl-N-[3-trifluoromethylphenyl]-1,3-benzothiazole-5-carboxamide

2-Methyl-1,3-benzothiazole-5-carboxylic acid (150 mg, 0.660 mmol) was mixed with 3-trifluoromethylaniline (209 mg, 1.30 mmol), EDC (249 mg, 1.30 mmol) and DMAP (158 mg, 1.30 mmol) in DCM (5.00 mL) and DMF (2.00 mL) for 18 hours. The mixture was concentrated, and the product was purified by flash chromatography on silica gel, eluting with mixtures of heptanes and EtOAc (95/5 to 50/25), to yield the product (58.1 mg, 0.173 mmol, 26.2%). ¹H NMR (600 MHz, CHLOROFORM-D) δ ppm 2.83 (s, 3H) 7.33 (d, J=7.94 Hz, 1H) 7.45 (t, J=7.94 Hz, 1H) 7.85 (d, J=7.94 Hz, 1H) 7.90-7.96 (m, 1H) 8.03 (t, J=8.19 Hz, 1H) 8.08 (s, 1H) 8.39 (s, 1H); MS [M+H] calcd. 337.0, found 337.0.

### Examples 6

### 2-methyl-N-[2-trifluoromethylbenzyl]-1,3-benzothiazole-5-carboxamide

2-Methyl-1,3-benzothiazole-5-carboxylic acid (150 mg, 0.660 mmol) was mixed with 2-trifluoromethylbenzylamine (228 mg, 1.30 mmol), EDC (249 mg, 1.30 mmol) and DMAP (158 mg, 1.30 mmol) in DCM (5.00 mL) and DMF (2.00 mL) for 18 hours. The mixture was concentrated, and the product was purified by flash chromatography on silica gel, eluting with mixtures of heptanes and EtOAc (95/5 to 50/25), to yield the product (123 mg, 0.350 mmol, 53.3%). ¹H NMR (600 MHz, MeOD) δ ppm 2.85 (s, 3H) 4.70 (s, 2H) 7.29-7.35 (m, 1H) 7.43-7.50 (m, 2H) 7.59 (d, J=7.68 Hz, 1H) 7.87-7.92 (m, J=8.71 Hz, 1H) 8.03 (d, J=8.45 Hz, 1H) 8.31 (s, 1H); MS [M+H] calcd. 351.0, found 351.0.

### Examples 7

### 2-methyl-N-[4-trifluoromethylbenzyl]-1,3-benzothiazole-5 carboxamide

2-Methyl-1,3-benzothiazole-5-carboxylic acid (150 mg, 0.660 mmol) was mixed with 4-trifluoromethylbenzylamine (228 mg, 1.30 mmol), EDC (249 mg, 1.30 mmol) and DMAP (158 mg, 1.30 mmol) in DCM (5.00 mL) and DMF (2.00 mL) for 18 hours. The mixture was concentrated, and the product was purified by flash chromatography on silica gel, eluting with mixtures of heptanes and EtOAc (95/5 to 50/25), to yield the product (114 mg, 0.325 mmol, 49.2%). ¹H NMR (600 MHz, MeOD) δ ppm 2.84 (s, 3H) 4.56 (s, 2H) 7.43 (d, J=7.94 Hz, 2H) 7.51 (d, J=8.19 Hz, 2H) 7.88 (d, J=8.45 Hz, 1H) 8.02 (dd, J=8.45, 2.30 Hz, 1H) 8.27-8.33 (m, J=1.02 Hz, 1H); MS [M+H] calcd. 351.0, found 351.0.

### Examples 8

### 2-methyl-N-[3-trifluoromethylbenzyl]-1,3-benzothiazole-5-carboxamide

2-Methyl-1,3-benzothiazole-5-carboxylic acid (150 mg, 0.660 mmol) was mixed with 3-trifluoromethylbenzylamine (228 mg, 1.30 mmol), EDC (249 mg, 1.30 mmol) and DMAP (158 mg, 1.30 mmol) in DCM (5.00 mL) and DMF (2.00 mL) for 18 hours. The mixture was concentrated, and the product was purified by flash chromatography on silica gel, eluting with mixtures of heptanes and EtOAc (95/5 to 50/50), to yield the product (131 mg, 0.370 mmol, 57.0%). ¹H NMR (600 MHz, MeOD)) δ ppm 2.84 (s, 3H) 4.56 (s, 2H) 7.43 (s, 2H) 7.53 (d, J=7.42 Hz, 1H) 7.56 (s, 1H) 7.87 (dd, J=8.45, 1.54 Hz, 1H) 8.01 (d, J=8.45 Hz, 1H) 8.29 (s, 1H); MS [M+H] calcd. 351.0, found 351.0.

### Examples 9

### N-4-methoxy-2-naphthyl-2-methyl-1,3-benzothiazole-5-carboxamide

2-Methyl-1,3-benzothiazole-5-carboxylic acid (200 mg, 1.03 mmol) was mixed with 4-methoxynaphthalen-2-amine (358 mg, 1.03 mmol), EDC (240 mg, 1.25 mmol) and DMAP (153 mg, 1.25 mmol) in DCM (10.0 mL) for 18 hours. The mixture was concentrated, and the product was purified by flash chromatography on silica gel, eluting with mixtures of heptanes and EtOAc (95/5 to 75/25), to yield the product (95.0 mg, 0.270 mmol, 27.0%).

¹H NMR (600 MHz, DMSO-D6) δ ppm 2.87 (s, 3H) 3.99 (s, 3H) 7.38-7.43 (m, 1H) 7.46 (d, J=2.05 Hz, 1H) 7.48-7.53 (m, 1H) 7.82 (d, J=8.19 Hz, 1H) 8.01-8.10 (m, 2H) 8.16 (d, J=4.86 Hz, 1H) 8.22 (d, J=8.45 Hz, 1H) 8.62 (d, J=1.28 Hz, 1H) 10.53 (s, 1H); MS [M+H] calcd. 349.0, found 349.0.

### Examples 10-21

The following examples were prepared by the general procedure of Example 1-9 using 2-Methyl-1,3-benzothiazole-5-carboxylic acid (Intermediate 1) and the appropriate amine as indicated in the below table.

| | | Mass | Mass | | |
|---|---|---|---|---|---|
| Ex. | Chemical name | calcd. | found | Proton NMR | Amine |
| 10 | N-(4- | 347.0 | 346.7 | (600 MHz, DMSO-D6) δ ppm | (4-bromophenyl)amine |
| | bromophenyl)-2- | | | 2.84 (s, 3H) 7.55 (d, J = 8.70 Hz, | |
| | methyl-1,3- | | | 2H) 7.80 (d, J = 8.70 Hz, 2H) | |
| | benzothiazole-5- | | | 7.96 (d, J = 8.19 Hz, 1H) | |
| | carboxamide | | | 8.19 (d, J = 8.45 Hz, 1H) | |
| | | | | 8.52 (s, 1H) 10.49 (s, 1H) | |
| 11 | 2-methyl-N-[2- | 311.1 | 311.0 | (600 MHz, DMSO-D6) δ ppm | [2-(4- |
| | (4- | | | 2.26 (s, 3H) 2.80-2.85 (m, 5H) | methylphenyl)ethyl]- |
| | methylphenyl)ethyl]- | | | 3.49 (q, J = 6.66 Hz, 2H) | amine |
| | 1,3- | | | 7.09-7.15 (m, 4H) 7.85 (d, | |
| | benzothiazole-5- | | | J = 8.45 Hz, 1H) 8.11 (d, J = 8.19 Hz, | |
| | carboxamide | | | 1H) 8.35 (s, 1H) 8.71 (s, 1H) | |
| 12 | N-[2-(3- | 315.1 | 315.0 | (600 MHz, DMSO-D6) δ ppm | [2-(3-fluorophenyl)ethyl]- |
| | fluorophenyl)ethyl]- | | | 2.83 (s, 3H) 2.90 (t, J = 7.17 Hz, | amine |
| | 2-methyl-1,3- | | | 2H) 3.54 (q, J = 6.91 Hz, 2H) | |
| | benzothiazole-5- | | | 7.01-7:06 (m, 1H) 7.10 (t, | |
| | carboxamide | | | J = 6.91 Hz, 2H) 7.31-7.36 (m, | |
| | | | | 1H) 7.84 (dd, J = 8.45, 1.28 Hz, | |
| | | | | 1H) 8.11 (d, J = 8.45 Hz, 1H) | |
| | | | | 8.34 (s, 1H) 8.72 (t, J = 5.12 Hz, | |
| | | | | 1H) | |
| 13 | N-(5-isopropoxy- | 377.1 | 377.0 | (400 MHz, DMSO-D6) δ ppm | 5-isopropoxynaphthalen- |
| | 1-naphthyl)-2- | | | 1.29 (d, J = 6.05 Hz, 6H) | 1-amine |
| | methyl-1,3- | | | 2.86 (s, 3H) 4.62-4.73 (m, 1H) | |
| | benzothiazole-5- | | | 7.21 (dd, J = 8.89, 2.44 Hz, 1H) | |
| | carboxamide | | | 7.28 (d, J = 2.15 Hz, 1H) | |
| | | | | 7.38 (t, J = 7.71 Hz, 1H) 7.55 (d, | |
| | | | | J = 7.23 Hz, 1H) 7.78 (d, J = 8.01 Hz, | |
| | | | | 1H) 7.90 (d, J = 8.98 Hz, 1H) | |
| | | | | 8.06 (dd, J = 8.30, 1.46 Hz, 1H) | |
| | | | | 8.22 (d, J = 8.40 Hz, 1H) | |
| | | | | 8.63 (s, 1H) 10.46 (s, 1H) | |
| 14 | 2-methyl-N-{2- | 365.1 | 365.0 | (400 MHz, CD3OD) δ ppm | {2-[4-(trifluoromethyl)- |
| | [4- | | | 2.85 (s, 3H) 3.03 (t, J = 7.23 Hz, | phenyl]ethyl}amine |
| | (trifluoromethyl) | | | 2H) 3.67 (t, J = 7.23 Hz, 2H) | |
| | phenyl]ethyl}- | | | 7.47 (d, J = 8.01 Hz, 2H) | |
| | 1,3- | | | 7.60 (d, J = 8.01 Hz, 2H) 7.79 (dd, | |
| | benzothiazole-5- | | | J = 8.40, 1.76 Hz, 1H) 8.00 (d, | |
| | carboxamide | | | J = 8.40 Hz, 1H) 8.28 (d, J = 1.76 Hz, | |
| | | | | 1H). | |
| 15 | N-[2-(4- | 325.1 | 325.0 | (400 MHz, CD3OD) δ ppm | [2-(4-ethylphenyl)ethyl]- |
| | ethylphenyl)ethyl]- | | | 1.19 (t, J = 7.62 Hz, 3H) | amine |
| | 2-methyl-1,3- | | | 2.59 (q, J = 7.62 Hz, 2H) 2.84 (s, 3H) | |
| | benzothiazole-5- | | | 2.90 (t, J = 7.42 Hz, 2H) | |
| | carboxamide | | | 3.60 (t, J = 7.42 Hz, 2H) | |
| | | | | 7.12 (d, J = 8.20 Hz, 2H) 7.17 (d, | |
| | | | | J = 8.20 Hz, 2H) 7.79 (dd, | |
| | | | | J = 8.40, 1.56 Hz, 1H) 7.99 (d, | |
| | | | | J = 8.40 Hz, 1H) 8.28 (d, J = 1.37 Hz, | |
| | | | | 1H). | |
| 16 | N-[2-(4- | 315.1 | 315.0 | (400 MHz, CD3OD) δ ppm | [2-(4-fluorophenyl)ethyl]- |
| | fluorophenyl)ethyl]- | | | 2.85 (s, 3H) 2.93 (t, J = 7.32 Hz, | amine |
| | 2-methyl-1,3- | | | 2H) 3.61 (t, J = 7.32 Hz, 2H) | |
| | benzothiazole-5- | | | 7.01 (ddd, J = 8.89, 6.64, 2.05 Hz, | |
| | carboxamide | | | 1H) 7.23-7.32 (m, 2H) | |
| | | | | 7.79 (dd, J = 8.40, 1.76 Hz, 2H) | |
| | | | | 7.99 (d, J = 8.40 Hz, 1H) | |
| | | | | 8.27 (d, J = 1.76 Hz, 1H). | |
| 17 | N-[2-(4-tert- | 353.1 | 353.0 | (400 MHz, CD3OD) δ ppm | [2-(4-tert-butylphenyl)- |
| | butylphenyl)ethyl]- | | | 1.29 (s, 9H) 2.85 (s, 3H) | ethyl]amine |
| | 2-methyl-1,3- | | | 2.90 (t, J = 7.42 Hz, 2H) 3.60 (t, | |
| | benzothiazole-5- | | | J = 7.42 Hz, 2H) 7.19 (d, J = 8.20 Hz, | |
| | carboxamide | | | 2H) 7.33 (d, J = 8.40 Hz, 2H) | |
| | | | | 7.80 (d, J = 8.40 Hz, 1H) | |
| | | | | 7.99 (d, J = 8.40 Hz, 1H) | |
| | | | | 8.29 (s, 1H). | |
| 18 | N-[2-(4- | 327.1 | 327.0 | (400 MHz, CD3OD) δ | [2-(4-methoxyphenyl)- |
| | methoxyphenyl)ethyl]- | | | ppm2.84 (s, 3H) 2.87 (t, | ethyl]amine |
| | 2-methyl- | | | J = 7.44 Hz, 2H) 3.58 (t, J = 7.32 Hz, | |
| | 1,3- | | | 2H) 3.74 (s, 3H) 6.84 (d, | |
| | benzothiazole-5- | | | J = 8.79 Hz, 2H) 7.17 (d, J = 8.59 Hz, | |
| | carboxamide | | | 2H) 7.79 (dd, J = 8.40, 1.76 Hz, | |
| | | | | 1H) 7.98 (d, J = 8.40 Hz, 1H) | |
| | | | | 8.27 (d, J = 1.37 Hz, 1H). | |
| 19 | N-(4- | 311.1 | 311.0 | (400 MHz, CD3OD) δ ppm | 4-isopropylaniline |
| | isopropylphenyl)- | | | 1.24 (d, J = 7.0 Hz, 6H) | |
| | 2-methyl-1,3- | | | 2.83-2.93 (m, 1H) 2.97 (s, 3H) | |
| | benzothiazole-5- | | | 7.19-7.26 (m, 2H) 7.55-7.63 (m, 2H) | |
| | carboxamide | | | 8.04 (dd, J = 1.7, 8.5 Hz, 1H) | |
| | | | | 8.15 (d, J = 8.6 Hz, 1H) | |
| | | | | 8.46 (d, J = 1.4 Hz, 1H) | |
| 20 | N-[2-(4- | 331.0 | 330.8 | (400 MHz, CD3OD) δ ppm | [2-(4-chlorophenyl)- |
| | chlorophenyl)ethyl]- | | | 2.86-2.95 (m, 5H) 3.61 (t, J = 7.3 Hz, | ethyl]amine |
| | 2-methyl-1,3- | | | 2H) 7.17-7.30 (m, 4H) | |
| | benzothiazole-5- | | | 7.84 (dd, J = 1.7, 8.5 Hz, 1H) | |
| | carboxamide | | | 8.06 (d, J = 8.6 Hz, 1H) | |
| | | | | 8.28 (d, J = 1.8 Hz, 1H) | |
| 21 | N-[2-(3,4- | 365.0 | 364.8 | (400 MHz, CD3OD) δ ppm | [2-(3,4-dichlorophenyl)- |
| | dichlorophenyl)ethyl]- | | | 2.86-2.96 (m, 5H) 3.61 (t, J = 7.1 Hz, | ethyl]amine |
| | 2-methyl- | | | 2H) 7.18 (dd, J = 2.2, | |
| | 1,3- | | | 8.2 Hz, 1H) 7.37-7.45 (m, 2H) | |
| | benzothiazole-5- | | | 7.84 (dd, J = 1.8, 8.4 Hz, 1H) | |
| | carboxamide | | | 8.06 (d, J = 8.6 Hz, 1H) | |
| | | | | 8.27 (d, J = 1.6 Hz, 1H) | |

### Examples 22

### N-4-tert-butylphenyl-2-hydroxymethyl-1,3-benzothiazole-5-carboxamide

2-Methyl-1,3-benzothiazole-5-carboxylic acid (430 mg, 1.89 mmol) and SeO₂ (628 mg, 5.65 mmol) were mixed in dioxane (50.0 mL) and heated to 100° C. for 18 hours. The mixture was evaporated to dryness and then dissolved in MeOH (10.0 mL). NaBH₄ (214 mg, 5.65 mmol) was added, and the mixture was stirred for 20 minutes. The mixture was evaporated to dryness, and the residue was dissolved in DCM (25.0 mL). AcCl (599 mg, 7.60 mL) was added, followed by Et₃N (769 mg, 7.60 mmol). The mixture was stirred for 30 minutes and then evaporated to dryness. The residue was dissolved in DCM (25.0 mL) and aniline (1.06 g, 11.3 mmol) and Et₃N (218 mg, 2.15 mmol) were added. The mixture was stirred for 30 minutes and then washed with a saturated solution of NaHCO₃ followed by 1N HCl. The organic phase was dried with Na₂SO₄, filtered and concentrated to yield a mostly pure compound (399 mg, 1.59 mmol, 84.0%). The resulting product, 2-hydroxymethyl-1,3-benzothiazole-5-carboxylic acid, (150 mg, 0.600 mmol) was mixed with the 4-tert-butylaniline (173 mg, 0.900 mmol), EDC (110 mg, 0.900 mmol) and DMAP (134 mg, 0.900 mmol) in DCM (5.00 mL) for 12 hours. The mixture was washed with a saturated solution of NaHCO₃, dried with Na₂SO₄, filtered and concentrated. The residue was dissolved in THF (3.00 mL), and a 1N solution of NaOH (3.00 mL) was added. The mixture was stirred for 1 hour, and then evaporated to dryness. The product was purified by flash chromatography on silica gel, eluting with mixtures of heptanes and EtOAc (80/20 to 50/50) to yield the product (43.1 mg, 0.130 mmol, 22.0% 2 steps). ¹H NMR (600 MHz, MeOD) δ ppm 1.22 (s, 9H) 4.90 (s, 2H) 7.30 (d, J=8.70 Hz, 2H) 7.50 (d, J=8.70 Hz, 2H) 7.89 (d, J=8.45 Hz, 1H) 8.04 (dd, 1H) 8.37 (d, J=1.28 Hz, 1H); MS calcd. [M+H] 341.0, found 341.0.

### Examples 23

### 2-(hydroxymethyl)-N-[2-(4-methylphenyl)ethyl]-1,3-benzothiazole-5-carboxamide

2-Methyl-N-[2-(4-methylphenyl)ethyl]-1,3-benzothiazole-5-carboxamide, example 11, (280 mg, 0.9 mmol) was dissolved in 10 mL of dioxane. Grounded selenium dioxide (485 mg, 4.37 mmol, 4.85 equiv) was added and the mixture heated in a sealed tube at 100° C. overnight. After cooling to room temperature, the mixture was filtered over Celite (rinced with methanol) and the filtrated evaporated to dryness. The residue was dissolved in. 15 mL of methanol, sodium borohydride (105 mg, 2.78 mmol, 3.1 equiv) was added in small portions and the mixture stirred for 20 min. Volatiles were evaporated, the residue was dissolved in ethyl acetate, washed with water, dried over magnesium sulfate, filtered and evaporated to dryness. The crude product was purified by reverse-phase HPLC (water acetonitrile 80:20 to 5:95) to yield the product (105 mg, 0.24 mmol, 27%) as the TFA salt. ¹H NMR (400 MHz, MeOD) δ ppm 2.29 (s, 3H) 2.89 (t, J=7.42 Hz, 2H) 3.60 (t, J=7.42 Hz, 2H) 4.97 (s, 2H) 7.10 (d, J=7.80 Hz, 2H) 7.15 (d, J=7.60 Hz, 2H) 7.81 (dd, J=8.40, 1.56 Hz, 1H) 8.06 (dd, J=8.40, 0.59 Hz, 1H) 8.30 (dd, J=1.76, 0.39 Hz, 1H); MS [M+H] calcd. 327.1, found 327.0.

### Examples 24

### N-[2-(3-fluorophenyl)ethyl]-2-(hydroxymethyl)-1,3-benzothiazole-5-carboxamide

The crude N-[2-(3-fluorophenyl)ethyl]-2-methyl-1,3-benzothiazole-5-carboxamide, example 12, (˜1 mmol) was dissolved in 10 mL of dioxane. Grounded selenium dioxide (485 mg, 4.37 mmol, 4.85 equiv) was added and the mixture heated in a sealed tube at 95° C. overnight. After cooling to room temperature, volatiles were evaporated and the residue was dissolved in 10 mL of methanol. Sodium borohydride (105 mg, 2.78 mmol, 3.1 equiv) was added in small portions and the mixture stirred for 20 min. Volatiles were evaporated, the residue was dissolved in ethyl acetate, washed with water, dried over magnesium sulfate, filtered and evaporated to dryness. The crude product was purified by reverse-phase HPLC (water acetonitrile 70:30 to 50:50) yielding the product (87 mg, 0.2 mmol, 20% global yield, including, preparation of example 12) as the TFA salt. ¹H NMR (400 MHz, MeOD) δ ppm 2.29 (s, 3H) 2.89 (t, J=7.42 Hz, 2H) 3.60 (t, J=7.42 Hz, 2H) 4.97 (s, 2H) 7.10,(d, J=7.80 Hz, 2H) 7.15 (d, J=7.60 Hz, 2H) 7.81 (dd, J=8.40, 1.56 Hz, 1H) 8.06 (dd, J=8.40, 0.59 Hz, 1H) 8.30 (dd, J=1.76, 0.39 Hz, 1H);); MS [M+H] calcd. 331.1, found 331.0.

### Pharmacology

1. hVR1 FLIPR (Fluorometric Image Plate Reader) screening assay

Transfected CHO cells, stably expessing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1×HBSS, 10 mM D-Glucose, 1 mM CaCl₂, 10 mM HEPES, 10×7.5% NaHCO₃ and 2.5 mM Probenecid).

### FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

### FLIPR (Fluorometric Image Plate Reader) screening assay with HEK T-REX hVR1.

HEK T-REX hVR1 inducible cells are grown in supplemented DMEM medium (10% FBS, 2 mM Glutamine, 5 μg/ml Blasticidine & 350 μg/ml Zeocin). HEK cells are plated in 384-black polylysine coated plate (Costar) at 10000 cells/well/50 μl for 24 hours or 5,500 cells/well 48 hours in a humidified incubator (5% CO₂ and 37° C.) in DMEM medium without selection agent. HEK T-Rex hVR1 cells are induced with 0.1 μg/ml Tetracycline 16 hours prior the experiment.

Subsequently, the media is removed from the cell plate by inversion and 2 μM Fluo-4 is added using a multidrop (Labsystems). Following the 30 to 40 minutes dye incubation in the dark at 37° C. and 2% CO₂, the extracellular dye present is washed away using an Microplate Washer Skatron Embla 384, leaving the cells in 25 μl of assay buffer (1×HBSS without Ca⁺⁺/Mg⁺⁺/sodium bicarbonate, 1 mM CaCl₂ & 5 mM D-Glucose).

### FLIPR Assay—IC₅₀ Determination Protocol

For IC₅₀ determinations the fluorescence is read using FLIPR filter 1 (em 520-545 nM). A cellular baseline recording is taken for 10 seconds, followed by 12.5 μl addition of test compounds, 10 points dilution 3 fold concentration, yielding cellular concentration ranging from 22.5 μM to 0.1 nM. Data are collected every 2 seconds for a further 5 minutes prior to the addition of a VR1 agonist solution: 20 nM (or 50 nM) capsaicin solution is added by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curie upon the addition of capsaicin. Maximum inhibition, Hill slope and IC₅₀ data for each compound are generated.

### List of Abbreviations

### VR1 vanilloid receptor 1

### IBS irritable bowel syndrome

### IBD inflammatory bowel disease

### GERD gastro-esophageal reflux disease

### HEPES 4-(2-Hydroxyethyl)piperazine-1-ethanesulfonic acid

### EGTA Ethylene glycol-bis(2-aminoethylether)-N,N,N′,N′-tetraacetic acid

### EMBLA Skatron, Plate Cell Washer, from Molecular Devices company

### FLIPR Fluorometric Image Plate Reader

### HBSS Hank's Balanced Salt Solution

### MES (2-[N-Morphholino]ethanesulfonic acid) Hydrate, Sigma cat# M-5287

### NUT Nutrient mixture F-12, medium for culturing cells

### MEM Minimal Eagle Medium

### Results

Typical IC₅₀ values as measured in the assays described above are 10 μM or less. In one aspect of the invention the IC₅₀ is below 500 nM.

Results from the hVR1 FLIPR

| Example No. | IC₅₀ nM (agonist) |
|---|---|
| 1 | 226 (capsaicin) |
| 7 | 2782 (capsaicin) |
| 8 | 1660 (capsaicin) |

1. A compound of formula I 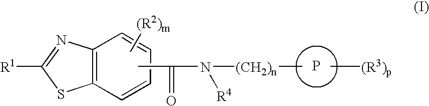
wherein:
ring P is C₆₋₁₀aryl, C₃₋₁₁cycloalkyl or C₅₋₁₀heteroaryl;
R¹ is H, C₁₋₄alkyl, hydroxyC₁₋₆alkyl, C₁₋₆alkylOC₀₋₆alkyl, COOC₀₋₆alkyl, NH₂, NHC₁₋₆alkyl, N(C₁₋₆alkyl)₂, NH(aryl) or N(aryl)₂;
R² is H, C₁₋₄alkyl, halo, hydroxyC₀₋₆alkyl or C₁₋₆alkylOC₀₋₆alkyl;
m is 0, 1, 2 or 3;
n is 0, 1, 2, 3, 4 or 5;
R³ is NO₂, NH₂C₀₋₆alkyl, halo, N(C₁₋₆alkyl)₂C₀₋₆alkyl, C₁₋₆alkyl, C₂₋₆alkenyl, C₂₋₆alkynyl, C₁₋₆haloalkyl, C₁₋₆haloalkylO, C₅₋₆arylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylNC₀₋₆alkyl, (C₀₋₆alkyl)₂NC(O)C₀₋₆alkyl, (C₀₋₆alkyl)₂OC(O)C₀₋₆alkyl or (C₀₋₆alkyl)₂C(O)OC₀₋₆alkyl;
p is 1, 2, 3, 4 or 5; and
R⁴ is H, C₁₋₆alkyl, arylC₀₋₆alkyl, C₁₋₆alkylOC₀₋₆alkyl or N(C₁₋₆alkyl)₂C₀₋₆alkyl;
or salts, solvates or solvated salts thereof.

2. A compound of formula Ib wherein R¹, R³, m and p, are as defined as in claim 1, and n is 0 and R² and R⁴ are H.

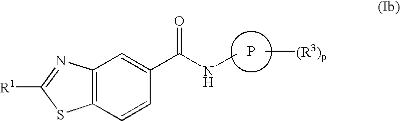

3. A compound of formula Ic, wherein R¹, R³, m and p, are as defined as in claim 1, and n is 1, 2, 3, 4 or 5 and R² and R⁴ are H.

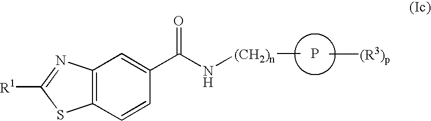

4. A compound according to claim 1 wherein ring P is phenyl.

5. A compound according to claim 1 wherein ring R¹ is methyl or hydroxyC₁₋₃alkyl.

6. A compound according to claim 1 wherein R³ is tert-butyl, phenyl, fluoromethyl, difluoromethyl or trifluoromethyl.

7. The compounds selected from the group consisting of
N-4-tert-butylphenyl-2-methyl-1,3-benzothiazole-5-carboxamide,
N-4-cyclohexylphenyl-2-methyl-1,3-benzothiazole-5-carboxamide,
2-methyl-N-[2-methyl-4-trifluoromethylphenyl]-1,3-benzothiazole-5-carboxamide,
2-methyl-N-[4-trifluoromethylphenyl]-1,3-benzothiazole-5-carboxamide,
2-methyl-N-[3-trifluoromethylphenyl]-1,3-benzothiazole-5-carboxamide,
2-methyl-N-[2-trifluoromethylbenzyl]-1,3-benzothiazole-5-carboxamide,
2-methyl-N-[4-trifluoromethylbenzyl]-1,3-benzothiazole-5-carboxamide,
2-methyl-N-[3-trifluoromethylbenzyl]-1,3-benzothiazole-5-carboxamide,
N-4-methoxy-2-naphthyl-2-methyl-1,3-benzothiazole-5-carboxamide,
N-4-tert-butylphenyl-2-hydroxymethyl-1,3-benzothiazole-5-carboxamide,
N-(4-bromophenyl)-2-methyl-1,3-benzothiazole-5-carboxamide,
2-methyl-N-[2-(4-methylphenyl)ethyl]-1,3-benzothiazole-5-carboxamide,
N-[2-(3-fluorophenyl)ethyl]-2-methyl-1,3-benzothiazole-5-carboxamide,
N-(5-isopropoxy-1-naphthyl)-2-methyl-1,3-benzothiazole-5-carboxamide,
2-methyl-N-{2-[4-(trifluoromethyl)phenyl]ethyl}-1,3-benzothiazole-5-carboxamide,
N-[2-(4-ethylphenyl)ethyl]-2-methyl-1,3-benzothiazole-5-carboxamide,
N-[2-(4-fluorophenyl)ethyl]-2-methyl-1,3-benzothiazole-5-carboxamide,
N-[2-(4-tert-butylphenyl)ethyl]-2-methyl-1,3-benzothiazole-5-carboxamide,
N-[2-(4-methoxyphenyl)ethyl]-2-methyl-1,3-benzothiazole-5-carboxamide,
N-(4-isopropylphenyl)-2-methyl-1,3-benzothiazole-5-carboxamide,
N-[2-(4-chlorophenyl)ethyl]-2-methyl-1,3-benzothiazole-5-carboxamide,
N-[2-(3,4-dichlorophenyl)ethyl]-2-methyl-1,3-benzothiazole-5-carboxamide,
N-4-tert-butylphenyl-2-hydroxymethyl-1,3-benzothiazole-5-carboxamide,
2-(hydroxymethyl)-N-[2-(4-methylphenyl)ethyl]-1,3-benzothiazole-5-carboxamide, and
N-[2-(3-fluorophenyl)ethyl]-2-(hydroxymethyl)-1,3-benzothiazole-5-carboxamide
or salts, solvates or solvated salts thereof.

8. A compound according to claim 1, for use in therapy.

9. A compound according to claim 1, in treatment of VR1 mediated disorders.

10. A compound according to claim 9 for treatment of acute and chronic pain, acute and chronic neuropathic pain and acute and chronic inflammatory pain.

11. A compound according to claim 9 for treatment of respiratory diseases.

12. A method of treatment of VR1 mediated disorders and for treatment of acute and chronic pain, acute and chronic neuropathic pain and acute and chronic inflammatory pain, and respiratory diseases, comprising administering to a mammal, including man in need of such treatment, a therapeutically effective amount of the compound of formula I, according to claim 1.

13. A pharmaceutical formulation comprising as active ingredient a therapeutically effective amount of the compound of formula I, according to claim 1, in association with one or more pharmaceutically acceptable diluents, excipients and/or inert carriers.

14. The pharmaceutical formulation according to claim 13, for use in the treatment of VR1 mediated disorders and for treatment of acute and chronic pain, acute and chronic neuropathic pain and acute and chronic inflammatory pain, and respiratory diseases.

15. A process for the preparation of the compound of formula I, wherein R¹ to R⁴, m, n and p, are defined as in claim 1, comprising; 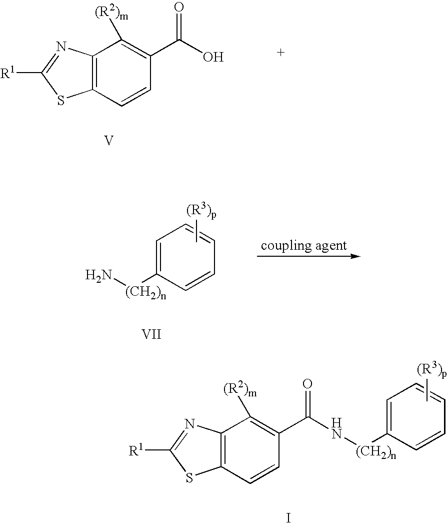
reaction of the carboxylic acids of formula V with aromatic amine of formula VII.

16. The compound 2-methyl-1,3-benzothiazole-5-carboxylic acid used as an intermediate in the preparation of a compound of formula I.
